(19) United States
(12) Patent Application Publication
(10) Pub. No.: US 2003/0124104 A1
(43) Pub. Date: Jul. 3, 2003

(54) Probiotic compositions

(76) Inventor: Sean Farmer, San Diego, CA (US)

Correspondence Address: MINTZ, LEVIN, COHN, FERRIS,, GLOVSKY AND POPEO, P.C., One Financial Center, Boston, MA, 02111, US

(21) Appl. No.: 10/287,904

(22) Filed: Nov. 5, 2002

Related U.S. Application Data
(60) Provisional application No. 60/338,785, filed on Nov. 5, 2001.

(51) Int. Cl. A61K 45/00; C12N 1/21

(52) U.S. Cl. 424/93.46; 424/93.462; 435/252.31

(57) ABSTRACT

The invention provides a bacterium which forms a spore that is germination-competent in the presence of a bile acid and methods of colonizing intestinal tissue of an animal by administering bacterial cells or spores to the animal

### RELATED APPLICATIONS

This application claims priority to provisional patent applications U.S. S. No. 60/338,785, filed Nov. 5, 2001, which is incorporated herein by reference in its entirety.

### BACKGROUND OF THE INVENTION

The invention relates to nutritional supplements.

Gastrointestinal microflora play a number of vital roles in maintaining gastrointestinal tract function and overall physiological health. Probiotics is a term, which refers to the use of microorganisms in a positive way to benefit health. Probiotic bacteria are ingested to enhance intestinal flora and aid in digestion. Such bacteria may also inhibit harmful strains of bacteria from multiplying. Common probiotic bacteria include Lactobacilli and Bifidobacteria, which are widely utilized in yogurts and other dairy products.

### SUMMARY OF THE INVENTION

The invention provides a Bacillus bacterium which forms a spore that is germination-competent in the presence of a bile acid. The bacterium is in the form of a vegetative cell, endospore, or mature spore. A vegetative cell is one which is capable of actively growing. An endospore or spore is a tough, dormant form of a bacterial cell that is resistant to desiccation, heat, and a variety of chemical and radiation treatments that are otherwise lethal to non-endospore bacterial cells. The endospore is the intracellular product of sporogenesis, and a spore is an endospore which has been released from a cell, i.e., it exists is a free state. Sporulation and sporogenesis refer to the formation of an endospore by a vegetative (i.e., growing) cell.

Spore germination is the transformation from an endospore/spore state to a vegetative state. By germination-competent is meant that a spore undergoes a transition from a dormant stage (non-replicating) to an actively-replicating vegetative stage. Bile acids generally inhibit spore germination; however the spores of the invention germinate in the presence of a bile acid. For example, the spore germinates in an environment in which the concentration of bile acid is greater than about 1,000 mg/liter, more preferably greater than about 10,000 mg/liter, more preferably greater than about 20,000 mg/liter, more preferably, greater than about 25,000 mg/liter, and most preferably, greater than about 30,000 mg/liter. The bile acid is preferably cholic acid, deoxycholic acid, and/or taurodeoxycholic acid. The bacterium, e.g., a Bacillus coagulans, Bacillus subtilis and Bacillus clausii, is in the form of a vegetative cell or a mature spore.

Also within the invention is a lactic acid-producing bacterium, e.g., member of the Sporolactobacillus species, which forms endospores that are germination-competent in the presence of a bile acid. For example, the spore germinates in an environment in which the concentration of bile acid is greater than about 1,000 mg/liter, more preferably greater than about 10,000 mg/liter, more preferably greater than about 20,000 mg/liter, more preferably, greater than about 25,000 mg/liter, and most preferably, greater than about 30,000 mg/liter. The bile acid is preferably cholic acid, deoxycholic acid, and/or taurodeoxycholic acid. The invention encompasses vegetative cells as well as the spores themselves. Spores successfully traverse the acidic environment of the stomach and germinate in the intestines in the presence of a bile acid, an environment that inhibits the germination of conventional spores. Following germination, the vegetative bacterial cells colonize the small and/or large intestine and aid in digestion and inhibit growth of pathogens.

Vegetative cells are formulated in a composition that protects the cells from being killed by the acid environment of the stomach. Cells formulated in this manner successfully traverse the stomach to colonize the small and/or large intestine. Accordingly, the invention includes a composition containing a Bacillus bacterium or a lactic acid-producing bacterium and a pharmaceutically-acceptable acid-resistant (“enteric”) carrier. Preferably, the vegetative cell is a Bacillus coagulans cell. By acid-resistant is meant that the carrier or coating does not dissolve in an acidic environment. An acidic environment is characterized by a pH of less than 7. The acid-resistant carrier is resistant to acids at pH less than about 4.0. Preferably, the carrier does not dissolve in pH 2-3. Most preferably, it does not dissolve in pH of less than 2. To protect vegetative bacterial cells from stomach acids, the cells are coated or encapsulated with the acid-resistant carrier. The composition optionally includes other components such as glucose and phosphoric acid or other nutrient compounds to boost bacterial growth after removal of the carrier or coating.

The spores and cells are useful as probiotics. Thus, the invention includes a method of colonizing an intestine of a mammal such as a human patient with a Bacillus bacterium or a spore-forming lactic-acid producing bacterium by administering to the mammal a Bacillus spore, which is germination-competent in the presence of a bile acid such as cholic acid, deoxycholic acid, and/or taurodeoxycholic acid. For example, the bacterium is a Bacillus coagulans, Bacillus subtilis, or Bacillus clausi bacterium. Small and/or large intestinal tissue is colonized. The invention also includes a method of colonizing an intestine of a mammal such as a human patient with a Bacillus bacterium or a spore-forming lactic-acid producing bacterium by administering to the mammal a vegetative bacterial cell formulated in an acid-resistant carrier.

One advantage of the invention is that viable vegetative cells effectively gain access to the small intestine where they rapidly multiply and colonize intestinal tissue to confer a clinical benefit. Another advantage is that the spores germinate into vegetative cells in an environment in which is inhibitory to conventional spores, thereby providing a mechanism for reliably colonizing intestinal tissue to confer clinical benefit. Unlike known probiotic bacterial preparations, the compositions of the invention reliably lead to colonization of the small and large intestine.

Other features and advantages of the invention will be apparent from the following detailed description and from the claims.

### DETAILED DESCRIPTION

Bile acids facilitate excretion of cholesterol, aid in absorption of dietary fats, and aid in water and electrolyte transport in the small bowel and colon. In humans, primary bile acids include cholic acid (cholate) and chenodeoxycholic acid. Secondary bile acids include deoxycholic acid (deoxycholate) and lithocholic acid (lithocholate). Other bile acids include ursodeoxycholic acid.

Some strains of bacteria are highly sensitive to these acids. The probiotic genera Lactobacillus and Bifidobacterium are relatively more sensitive than other bacterial species. Bacillus vegetative cells are generally resistant to these acids.

Bacillus, rather than Lactobacillus, is administered to mamals for intestinal colonization because the endospores produced by Bacillus are highly resistant to the gastric acid in the stomach that most often results in the death of the Lactobacillus vegetative cell. Some Bacillus cells die in this acid environment as well. Prior to the invention, it was thought that the Bacillus endospore remained viable throughout the digestive process and relocated to the small intestine where it would germinate and form a new vegetative cell. Evidence now indicates that Bacillus spores seldom, if ever, germinate in the small or large bowel, because sub-acute levels of cholic acids (e.g., deoxycholic and tauro-deoxycholic acid) inhibit spore germination.

Minimum inhibitory concentration (MIC) dilutions for typical colonic bacteria indicate that Bacillus bacteria/spores exhibit very high sensitivity to cholic acids compared to other bacteria (Table 1).

TABLE 1

| | MIC TDOC | | MIC of DOC | |
|---|---|---|---|---|
| Species | μg/mL (mM) | | μg/mL (mM) | |
| B. subtilis | 195 | (0.4) | 78 | (0.2) |
| B. clausii | 24 | (≦0.05) | 78 | (0.2) |
| S. pneumoniae | 25,000 | (51.2) | 20 | (0.05) |
| E. faecalis | 25,000 | (51.2) | 625 | (1.6) |
| E. Faecium | 3,125 | (6.4) | 625 | (1.6) |
| S. aureus | ≧25 000 | (≧51.2) | ≧20 000 | (≧51.2) |
| E. coli | ≧25 000 | (≧51.2) | ≧20 | (≧0.05) |
| K. pneumoniae | ≧25 000 | (≧51.2) | ≧20 | (≧0.05) |
| P. mirabilils | ≧25 000 | (≧51.2) | ≧20 | (≧0.05) |

The bile acid sensitivity data shown in Table 1 indicate that bowel colonization after ingestion of enteric pathogens is unhindered by colonic cholic acids. In contrast, Bacillus based probiotics are often bile resistant. Bacillus vegetative cells are very bile acid resistant, but conventional Bacillus endospores are sensitive to low concentration of bile acids, i.e., spore germination and/or rehydration is inhibited by the presence of even low concentrations of bile acids.

Animal Feed Supplements

Bacillus bacteria are used in animal feed to enhance weight gain, aid in digestion by production of various enzymes, and control the growth of pathogens. As with humans, bile acids in other animals also inhibit spore germination. For example, only 50% of the Bacillus coagulans cells fed to chickens actually colonized the gut of the animals. Although still confering a benefit, this survival rate limits the cost effectiveness of Bacillus coagulans based probiotic feed supplementation. The compositions of the invention substantially improve the survival rate of the administered bacteria, thereby reducing the cost of therapy. To achieve the same or similar beneficial effect (weight gain, improved digestion, or inhibition of pathogenic bacterial species), the cost of supplementing feed with the compositions described herein is comparable or less expensive than antibiotic treatment.

Inhibition of Enteric Pathogens

The bile acid-resistant spores and enterically-coated vegetative cells described herein are also useful in inhibiting the growth of enteric pathogens such as vancomycin resistant enterococci (VRE) in humans and other susceptible animals. There is little or no benefit in administering conventional Bacillus spores with the intent of digestive colonization because the spores fail to germinate in the digestive tract. The invention solves this problem in two ways: (1) by providing spores that are resistant to the inhibitory activity of bile acids, and as a result, germinate into vegetative cells, which then colonize the colon, and (2) providing a vegetative bacterial cell, which is coated to allow passage through the stomach to the colon.

In a study conducted to ascertain the role of Bacillus species in reducing the colonization potential of vancomycin resistant enterococcus in humans, conventional Bacillus coagulans spores in the form of a spray-dried powder were re-suspended in sterile saline and then feed by feeding tube to mice. No change in the density of fecal enterococcus was observed after treatment, and little or no Bacillus coagulans was detected in stool samples. The spray-dried powder that was utilized in the study was a beige to off-white material that contained Bacillus coagulans spores.

Another formulation of Bacillus coagulans was tested in the mice using the same battery of tests. The second formulation contains germinated spores. The Bacillus coagulans spray-dried powder was used to inoculate one 250 ml Erlenmyer flask of Tryptic Soy Broth (TSB). The flask was then inserted into a orbital shaker at 37° C. for 3 days. The broth with the Bacillus biomass was then centrifuged and the pellet was resuspended in saline. This material (containing vegetative cells derived from germinated spores) was refed to the mice at the same dosage (in colony forming units) as was the previous spray-dried powered. The results of this testing indicated that 65% of the mice in the treatment group had experienced a 6 fold reduction in VRE colonization and the remaining 35% of the mice had no detectable VRE in their respective stool samples. The data indicated that when the spores of the spray-dried powder were germinated in a flask using TSB prior to administration to the animals, the germinated spores grew to a high vegetative cell density and were isolated from fecal samples from test animals. But, when the spray-dried powder alone was resuspended in saline and then fed by tube, there was no colonization and no isolation from stool samples. Human clinical studies showed the same results using a spray-dried powder. The data provides an explanation for the observation that Bacillus vegetative cells are rarely isolated from fecal samples and why Bacillus is considered a non-resident bacterium in humans and animals.

After ingestion, Bacillus may also colonize other areas of the body (such as the lymph nodes or spleen) in order to produce a benefit to the host. Ingestion of the lyophilized or spray-dried fermentation broth from a Bacillus culture is also beneficial. Such processed fermentation broth contains organic acids, bacteriocins, enzymes, and other bacterial components. These components are secreted into the broth or are release upon cell lysis.

Although some small percentage (e.g., 1%) of conventional Bacillus spores may germinate in the gut, these germinated spores go through few replications and fail to effectively compete with pathogens that are found in the colon. The spores and vegetative cells described herein result in rapid and reliable germination of spores and colonization of the colon with high numbers of vegetative cells.

Administration of Bacterial Cells and Spores

Vegetative bacterial cells and endospores are administered at a dose of 10,000-10¹¹ cells per administration. A typical therapeutic composition of the present invention contains in a one gram dosage formulation, from approximately 1×10³ to 1×10¹², and preferably approximately 2×10⁵ to 1×10¹⁰, colony forming units (CFU) of viable Bacillus bacteria (i.e., vegetative bacteria) or bacterial spores. Animals are treated daily, every three days, or every 5 days. Bacteria remain in and colonize the colon for a period of 3-5 days post-administration.

### EXAMPLE 1

### Endospore Germination in the Presence of a Bile Acid

Bacillus spores are selected for the capability of germinating in the presence of bile acids such as cholic, deoxycholic and tauro-deoxycholic acids. Bacillus coagulans is grown on Petri dishes with sub-inhibitory levels of cholic acid(s). Colonies resulting from germinated spores are isolated. Successive Petri dishes with higher concentrations of cholic acids are reinoculated until colonies, which produce cholic acid resistant spores, are obtained. The following Bacillus and related bacterial species are selected for endospore germination in the presence of a bile acid: Bacillus subtilis, Bacillus laterosporus, Bacillus coagulans, Bacillus megaterium, Bacillus polymyxa, Bacillus laevolacticus, Bacillus racemilacticus, Bacillus polyfermenticus, Bacillus clausii, Sporolactobacillus inulinus, Sporolactobacillus p44. The starting bacterial cultures are commercially available, e.g., from the American Type Culture Collection (ATCC).

To enhance sporulation, manganese sulfate (approximately 1 g/l) is added to the bacterial culture. Sporulation is also enhanced by starving the culture at mid log stage. The spore-containing cultures are then spray-dried and packaged into capsules or tablets for oral administration. Methods of making mature spore stocks are known in the art, e.g., freeze-drying, fluidized bed-drying, and spray-drying. A spore formulation may be dried at temperatures up to 60° C. without appreciable loss of viable spores.

### EXAMPLE 2

### Enterically-Coated Vegetative Bacillus Cells

Bacillus vegetative cells (with or without mature spores or endospores) are grown in broth culture using standard methods. The cells are recovered from fermentation vessels and made into a paste or dope. The vegetative cell formulation is not spray-dried. The dope contains nutrients, vitamins and amino acids to give a boost to initial growth. The cell-containing dope is encapsulated with an acid-resistant carrier or coating (enteric coating) that assures the survival of the cells through gastric acidity.

The enteric coating is pH-sensitive. The coating dissolves after the pH is greater than 4.0. For example, the coating dissolves in a neutral environment as is encountered in the small intestine, and does not dissolve in an acidic environment as is encountered in the stomach. Alternatively, the enteric coating dissolves when exposed to specific metabolic event such as an encounter with a digestive enzyme that is found in the small intestine. For example, the coating is digested by a pancreatic enzyme such as trypsin, chymotrypsin, or a pancreatic lipase. The formulation is hydrated in the small intestine. Digestion or dissolution of the coating allows liberation of vegetative bacterial cells, e.g., Bacillus cells, which colonize the intestine.

Vegetative cells are stabilized in a gel or paste such as an anhydrous carbohdrate paste. In alternate formulations, the cells are lyophillized and/or suspended in a gel or paste to render the cells dormant until they reach the small intestine. Enteric coating materials are known in the art, e.g., malic acid-propane 1,2-diol. Cellulose derivatives, e.g., cellulose acetate phthalate or hydroxypropyl methylcellulose phthalate (HPMCP), are also useful in enteric acid-resistant coatings. Other suitable enteric coatings include cellulose acetate phthalate, polyvinyl acetate phthalate, hydroxypropylmethylcellulose phthalate and anionic polymers of methacrylic acid and methyl methacrylate. Another suitable enteric coating is a water emulsion of ethylacrylate methylacrylic acid copolymer, or hydroxypropyl methyl cellulose acetate succinate (HPMAS). (See, e.g., U.S. Pat. No. 5,591,433). An enteric coating is designed to resist solution in the stomach and to dissolve in the neutral or alkaline intestinal fluid.

In some cases, a spore shock is required for optimal spore germination. Spores are shocked in a variety of standard methods, e.g, osmotic shock, heat shock, deprivation of nutrients, and/or exposure to certain acids. Without a spore shock, many Bacillus spores are unable to germinate and thus pass through the entire digestive system without offering any benefit. By administering vegetative Bacillus cells in an enteric coating (rather than conventional spores), one can overcome the spore-shock and bile acid obstacles that severely curtail the use of Bacillus based formulations in human nutrition.

Other embodiments are within the following claims.

What is claimed is:

1. A Bacillus bacterium, wherein said bacterium forms a spore, said spore being germination-competent in the presence of a bile acid.

2. The bacterium of claim 1, wherein said bacterium is in the form of a vegetative cell.

3. The bacterium of claim 1, wherein said bacteria is selected from the group consisting of Bacillus coagulans, Bacillus subtilis and Bacillus clausii.

4. The bacterium of claim 1, wherein said bacteria is Bacillus coagulans.

5. The bacterium of claim 1, wherein the concentration of said bile acid is greater than about 1,000 mg/liter.

6. The bacterium of claim 1, wherein the concentration of said bile acid is greater than about 10,000 mg/liter.

7. The bacterium of claim 1, wherein the concentration of said bile acid is greater than about 20,000 mg/liter.

8. The bacterium of claim 1, wherein the concentration of said bile acid is greater than about 25,000 mg/liter.

9. The bacterium of claim 1, wherein the concentration of said bile acid is greater than about 30,000 mg/liter.

10. The bacterium of claim 1, wherein said bile acid is selected from the group consisting of cholic acid, deoxycholic acid, and taurodeoxycholic acid.

11. A lactic acid-producing bacterium, wherein said bacterium forms a spore, said spore being germination-competent in the presence of a bile acid.

12. The bacterium of claim 11, wherein said bacterium is in the form of a vegetative cell.

13. The bacterium of claim 11, wherein said bacterium is a member of the Sporolactobacillus species.

14. The bacterium of claim 11, wherein the concentration of said bile acid is greater than about 1,000 mg/liter.

15. The bacterium of claim 11, wherein the concentration of said bile acid is greater than about 10,000 mg/liter.

16. The bacterium of claim 11, wherein the concentration of said bile acid is greater than about 20,000 mg/liter.

17. The bacterium of claim 11, wherein the concentration of said bile acid is greater than about 25,000 mg/liter.

18. The bacterium of claim 11, wherein the concentration of said bile acid is greater than about 30,000 mg/liter.

19. The bacterium of claim 11, wherein said bile acid is selected from the group consisting of cholic acid, deoxycholic acid, and taurodeoxycholic acid.

20. A Bacillus spore, wherein said spore is germination-competent in the presence of a bile acid.

21. The spore of claim 20, wherein said bile acid is selected from the group consisting of cholic acid, deoxycholic acid, and taurodeoxycholic acid.

22. The spore of claim 20, wherein the concentration of said bile acid is greater than about 1,000 mg/liter.

23. The spore of claim 20, wherein the concentration of said bile acid is greater than about 10,000 mg/liter.

24. The spore of claim 20, wherein the concentration of said bile acid is greater than about 20,000 mg/liter.

25. The spore of claim 20, wherein the concentration of said bile acid is greater than about 25,000 mg/liter.

26. The spore of claim 20, wherein the concentration of said bile acid is greater than about 30,000 mg/liter.

27. A composition comprising a lactic acid-producing bacterium and a pharmaceutically-acceptable acid-resistant carrier, wherein said acid-resistant carrier is resistant to acids at pH less than about 4.0.

28. The composition of claim 27, wherein said bacterium is coated with said acid-resistant carrier.

29. The composition of claim 27, wherein said bacterium is in the form of a vegetative cell.

30. The composition of claim 27, further comprising glucose and phosphoric acid.

31. A composition comprising a bacterium and a pharaceutically-acceptable acid-resistant carrier, wherein said bacterium is a Bacillus, and wherein said acid-resistant carrier is resistant to acids at pH less than about 4.0.

32. The composition of claim 31, wherein said bacterium is coated with said acid-resistant carrier.

33. The composition of claim 31, wherein said bacterium is in the form of a vegetative cell.

34. The composition of claim 31, wherein said bacterium is Bacillus coagulans.

35. The composition of claim 31, further comprising glucose and phosphoric acid.

36. A method of colonizing an intestine of a mammal with a Bacillus bacterium, comprising administering to said mammal a Bacillus spore, wherein said spore is germination-competent in the presence of a bile acid.

37. The method of claim 36, wherein said bile acid is selected from the group consisting of cholic acid, deoxycholic acid, and taurodeoxycholic acid.

38. The method of claim 36, wherein said bile acid is cholic acid.

39. The method of claim 36, wherein said bile acid is deoxycholic acid.

40. The method of claim 36, wherein said bile acid is taurodeoxycholic acid.

41. The method of claim 36, wherein said Bacillus is Bacillus coagulans.

42. The method of claim 36, wherein said Bacillus is Bacillus subtilis.

43. The method of claim 36, wherein said Bacillus is Bacillus clausi.

44. The method of claim 36, wherein said intestine is the small intestine.

45. The method of claim 36, wherein said intestine is the large intestine.

46. A method of colonizing an intestine of a mammal with a Bacillus bacterium, comprising administering to said mammal a composition comprising a vegetative Bacillus cell, wherein said composition comprises a pharmaceutically acceptable acid-resistant carrier, said acid-resistant carrier being resistant to acids at pH less than 4.0.

47. The method of claim 46, wherein said Bacillus is Bacillus coagulans.

48. The method of claim 46, wherein said Bacillus is Bacillus subtilis.

49. The method of claim 46, wherein said Bacillus is Bacillus clausi.

50. The method of claim 46, wherein said intestine is the small intestine or the large intestine.
